(19) United States
(12) Patent Application Publication
(10) Pub. No.: US 2022/0033463 A1
(43) Pub. Date: Feb. 3, 2022

(54) Preparation and Application of Soluble Tim-3 Recombinant Protein and Mutant Protein Thereof

(71) Applicant: Zhejiang University, Hangzhou (CN)

(72) Inventors: Ying Yang, Hangzhou (CN); Zhi Chen, Hangzhou (CN); Haihong Zhu, Hangzhou (CN); Yanning Liu, Hangzhou (CN); Guohua Lou, Hangzhou (CN); Yu Shi, Hangzhou (CN)

(21) Appl. No.: 17/506,676

(22) Filed: Oct. 21, 2021

Related U.S. Application Data
(63) Continuation of application No. PCT/CN2020/099600, filed on Jun. 30, 2020.

(30) Foreign Application Priority Data
Oct. 24, 2019 (CN) 201911019526.3

(51) Int. Cl. C07K 14/705

(52) U.S. Cl. CPC C07K 14/70503; A61K 38/00

(57) ABSTRACT

The present application belongs to the field of biotechnology, and particularly relates to preparation and use of soluble Tim-3 recombinant protein and a mutant protein thereof. The soluble Tim-3 recombinant protein is used to prepare a drug with the function of regulating monocytes or a drug with the function of enhancing tumor immune response, and the amino acid sequence of the soluble Tim-3 recombinant protein is as shown in SEQ ID NO: 1. In addition, the present application further provides the mutant proteins of the sTim-3 recombinant protein and preparation and use of the mutant protein. The mutant recombinant protein is obtained by screening through a directed evolutionary technology, with the function of regulating monocytes or the function of enhancing tumor immune response, and combines with a host codon optimization method to improve the expression efficiency.

### CROSS REFERENCE TO RELATED APPLICATION

The present application is a continuation application of PCT application No. PCT/CN2020/099600 filed on Jun. 30, 2020, which claims the benefit of Chinese Patent Application No. 201911019526.3 filed on Oct. 24, 2019. The contents of the above applications are incorporated herein by reference in their entirety.

### REFERENCE TO SEQUENCE LISTING

The Sequence Listing is submitted as an ASCII formatted text file via EFS-Web, with a file name of “SEQUENCE_LISTING.TXT”, a creation date of Oct. 20, 2021, and a size of 34,612 bytes. The Sequence Listing filed via EFS-Web is part of the specification and is incorporated in its entirety by reference herein.

### FIELD OF THE INVENTION

The present application belongs to the field of biotechnology, and particularly relates to preparation and use of soluble Tim-3 recombinant protein and a mutant protein thereof.

### BACKGROUND OF THE INVENTION

Biotechnological drugs mainly comprise proteins, peptides and nucleic acid molecules, covering almost hundreds of diseases including cancer, autoimmune diseases, infectious diseases, etc. Recombinant protein products made by protein engineering technology play an indispensable role in people's production, life and medical treatment. The recombinant protein drugs are the leading type of biotechnological drugs at present. Compared with small molecule chemical drugs, the recombinant protein drugs have a series of advantages, such as good efficacy, slight side effects and clear biological functions.

Liver failure is serious liver damage caused by a variety of factors, with a group of clinical symptoms mainly manifested by blood coagulation disturbances, jaundice, hepatic encephalopathy, ascites and the like. The incidence is rapid, the fatality rate is high, and the harm is great. It is globally recognized as a very challenging clinical critical disease that endangers the life of patients. At present, there is still a lack of effective drugs and means. Liver transplantation is currently considered to be the most effective method for the treatment of this disease, but its clinical application is greatly limited due to the shortage of donor livers and the high cost of treatment. Overactivity of monocytes of innate immune cells plays an important role in the pathogenesis of liver failure. Tim-3 (T cell immunoglobulin- and mucin-domain-containing molecule-3) on cell surfaces is expressed in the monocytes, macrophages, dendritic cells, lymphocytes and the like. Soluble Tim-3 (sTim-3) produced by shedding of Tim-3 molecules on cell membranes of the monocytes is negatively correlated with the expression of IL-12 and TNF-α in patients with sepsis. The previous in vitro studies of the research group found that sTiM-3 can decrease the expression level of TNF-α in primary human monocytes stimulated by LPS, which indicates that sTim-3 has the effect of inhibiting the activation of the monocytes.

At present, patients with liver cancer account for 4% of newly discovered patients with malignant tumors in the world every year, and liver cancer has become the second cause of tumor death in China, with a very high incidence rate and fatality rate. At present, surgical resection has been the main method of treatment, but the prognosis of surgical treatment is poor, and it is easy to relapse. Studies show that long-term low immunity is also one of the important causes of cancer, and improving the body's immune level is a key means of tumor treatment. Therefore, it is very necessary to search for anti-cancer drugs that can improve the immunity level. Tumor immunotherapy is the general term for a number of therapeutic approaches, including immune check point therapy, cytokine therapy, tumor vaccines and cell therapy. Research on tumor immune check point molecules mainly focuses on three molecules of Tim-3, CTLA-4 and PD1 which inhibit the activity of immune cells in microenvironments. Tumor immune check point inhibitors are the most important aspect of tumor immunotherapy at present, which can mobilize the function of the autoimmune system to eliminate tumors by inhibiting the immune escape of tumor cells. The discovery of Tim-3 molecules stems from the search for surface markers that distinguish Th1 cells from Th2 cells. A recent study found that the expression levels of soluble Tim-3, CTLA-4 and other indicators in tumor-bearing mice at different time periods are measured by a semi-quantitative RT-PCR method, and meanwhile tumor growth is measured. The results show that tumor growth is positively correlated with the expression of CTLA-4 and negatively correlated with the expression of soluble Tim-3, which suggests that soluble Tim-3 may have an inhibitory effect on tumor growth. No study has reported the role of sTim-3 in liver cancer at present. Recent studies on the cell-mediated immune response to HIV vaccines show that soluble PD1 and soluble Tim-3 enhance the ability of adenovirus vector SIV vaccines (rAd5-SIV) on the proliferation of mouse T cells, and more antigen-specific IFN-γ(+)CD4(+) and CD8(+) T cells are produced. It is suggested that sTim-3 may act as an immune adjuvant to enhance the T cell immune response of the body.

### SUMMARY OF THE INVENTION

The present application provides use and a purifying method of a novel soluble Tim-3 recombinant protein.

Use of soluble Tim-3 recombinant protein in preparing a drug with the function of regulating monocytes or a drug with the function of enhancing tumor immune response, and the amino acid sequence of the soluble Tim-3 recombinant protein is as shown in SEQ ID NO:1 (the soluble Tim-3 recombinant protein includes or does not include a His tag sequence, such as HHHHHH (SEQ ID NO:8); if the His tag sequence, such as HHHHHH (SEQ ID NO:8), is included, the tag sequence is preceded by a linker peptide, such as (G) nS, n=1-4, preferably n=4), i.e., GS, GGS, GGGS (SEQ ID NO:9), GGGGS(SEQ ID NO:10).

The drug with the function of regulating monocytes can be used for inhibiting overactivity of the monocytes in patients with inflammation, such as patients with liver failure, and the drug with the function of enhancing tumor immune response can be used for enhancing immune response in cancer patients, such as liver cancer patients, to avoid host's immune escape.

The concentration of the solubleTim-3 recombinant protein is preferably 80 ng/ml.

A purification method of soluble Tim-3 recombinant protein includes the following steps: performing fluid exchanging on cell culture solutions of eukaryotic CHO cells, prokaryotic E. coli and insect baculovirus expression system containing recombinant human soluble Tim-3 through microfiltration clarification and ultrafiltration concentration, and then passing through cation and anion chromatographic column, molecular sieve chromatographic column, hydrophobic chromatographic column and/or affinity chromatographic column, to obtain the recombinant protein with enough purity (purity determined by SDS-PAGE is >96%), wherein the amino acid sequence of the sTim-3 recombinant protein is as shown in SEQ ID NO: 1.

In addition, the present application further provides a mutant protein of the soluble Tim-3 recombinant protein and preparation and use of the mutant protein. The mutant recombinant protein is obtained by screening through a directed evolutionary technology, with the function of regulating monocytes or enhancing tumor immune response, and combines with a host codon optimization method to improve its expression efficiency.

A soluble Tim-3 recombinant mutant protein, which is a protein obtained in the mode that: on the basis of a gene sequence corresponding to human Tim-3 (NP_116171.3) extracellular domain amino acid (SEQ ID NO: 1), mutant genes are obtained by screening through a directed evolutionary technology, and then expressed, wherein the mutant sites of the mutant genes do not involve conserved sequences among species, but occur in non-conservative sequence positions.

Further, the soluble Tim-3 recombinant protein includes or does not include a His tag sequence, such as HHHHHH (SEQ ID NO:8); if the His tag sequence, such as HHHHHH (SEQ ID NO:8), is included, the tag sequence is preceded by a linker peptide, such as (G)nS, n=1-4, preferably n=4, i.e. i.e., GS, GGS, GGGS (SEQ ID NO:9), GGGGS(SEQ ID NO:10).

Furtherer, the gene sequence corresponding to the amino acid sequence can be optimized according to the characteristics of codons of hosts such as prokaryotes and eukaryotes.

Preferably, the amino acid sequence of the soluble Tim-3 recombinant mutant protein is one of sequences as shown in SEQ ID NOs: 2-7.

A preparation method of the soluble Tim-3 recombinant mutant protein, wherein performing mutant amplification screening to obtain the gene sequence using PCR kit, wherein the mutant protein can preserve conserved amino acid sequences from different species; and obtaining the mutant protein through the directed evolutionary technology on the basis of the gene sequence corresponding to the human Tim-3 (NP_116171.3) extracellular domain amino acid (SEQ ID NO: 1), and then expressing amino acids into the sTim-3 recombinant mutant protein with the preferable amino acid sequences being SEQ ID NOs: 2-7.

The expression of amino acid means that a pre-constructed recombinant protein gene expression vector performs expression in eukaryotic expression system, prokaryotic expression system and insect baculovirus expression system to extract and produce long-acting recombinant drugs.

Use of the soluble Tim-3 mutant protein in preparing a drug with the function of regulating monocytes or a drug with the function of enhancing tumor immune response. Preferably, the amino acid sequence of the soluble Tim-3 recombinant mutant protein is one of sequences as shown in SEQ ID NOs: 2-7.

The function of the drug is realized through regulating monocytes or enhancing tumor immune response. The drug with the function of regulating monocytes can be used for inhibiting overactivity of the monocytes in patients with inflammation, such as patients with liver failure, and the drug with the function of enhancing tumor immune response can be used for enhancing immune response in cancer patients, such as liver cancer patients, to avoid host's immune escape.

The present application constructs the soluble Tim-3 recombinant protein gene sequence into prokaryotic, eukaryotic and insect baculovirus expression vectors, and then the recombinant protein expressed by the host is purified into the soluble Tim-3 recombinant protein through a combination of various purification methods such as ion exchange chromatography, affinity chromatography and hydrophobic chromatography.

In order to obtain the mutant recombinant protein with stronger biological functions, a large number of mutations in the recombinant protein are screened by the directed evolution technology, and PCR amplification is performed by using GeneMorph II random mutation PCR kit (stratagene), and target genes are constructed into eukaryotic expression vectors, prokaryotic expression vectors or insect baculovirus expression vectors, and then mutant recombinant expression vector is transformed into a eukaryotic, prokaryotic or insect host for expression.

In order to obtain mutant recombinant expression plasmids with higher expression efficiency, codon optimization is performed through a host codon optimization technology. Specifically, according to the characteristics of host's eukaryotic, prokaryotic or insect codons, the site-directed mutation is performed by recombinant PCR technology to obtain a codon-optimized target sequence, then the target sequence is digested to be cloned to the corresponding expression vectors, and then the mutant recombinant expression vectors are transformed into the eukaryotic, prokaryotic or insect host for expression.

The soluble Tim-3 and the mutant recombinant protein are expressed and purified through a combination of various purification methods such as affinity chromatography, ion exchange chromatography and hydrophobic chromatography, and thereby the soluble Tim-3 recombinant protein and the mutant recombinant protein are purified. The principle of metal-chelate affinity chromatography is that some special amino acids on the surface of the protein interact with metal ions, thereby performing affinity purification on the protein, wherein a fusion tag 6×His-Tag with a combination of six His residues is relatively common and has the advantages of simple ligands, large adsorbing capacity, mild separation conditions, high universality, etc. Ion chromatography can not only carry out crude purification to effectively remove most of electrically charged host proteins and nucleic acids, but also achieve fine purification to remove trace impure proteins. Hydrophobic chromatography can not only enrich proteins but also remove most of pigment substances.

The present application discloses a preparation method of a soluble Tim-3 recombinant protein and its mutant protein in the technical field of bioengineering and their uses in diseases such as liver diseases (liver failure and liver cancer). The soluble Tim-3 recombinant protein is an extracellular secreted and soluble recombinant protein with small molecular weight. The recombinant protein contains or does not contain derivative protein with 6×His tag from Tim-3 extracellular soluble region. As for the mutant protein, the amino acid sequence containing a conservative structural region in evolution and optimized by host codons is obtained through the directed evolutionary technology. This type of recombinant protein has the function of regulating monocytes or the function of enhancing tumor immune response.

### BRIEF DESCRIPTION OF THE DRAWINGS

FIG. 1 shows SDS-PAGE identification of culture media, natural proteins, denatured proteins and purified proteins in Embodiment 1.

FIG. 2 shows the dose-dependent effect of soluble Tim-3 on TNF-α secretion of monocytes in Embodiment 2.

FIG. 3 shows the inhibitory effect of soluble Tim-3 on HMGB1 secretion of the monocytes in Embodiment 2.

FIG. 4 shows expression levels of mTim-3 and sTim-3 of the monocytes of patients with acute on chronic liver failure in embodiment 3.

FIG. 5 shows liver histology of mice with D-GalN/LPS acute liver failure improved by sTiM-3 in Embodiment 3.

FIG. 6 shows the effect of mutant and non-mutant sTim-3 on TNF-α secretion of the monocytes.

FIG. 7 shows amino acid sequences containing His and linker peptides.

### DETAILED DESCRIPTION OF THE EMBODIMENTS

In order to illustrate the present application more clearly, a further explanation of the present application is given below in combination with preferred embodiments and accompanying drawings. Those skilled in the art should understand that the following content described below is illustrative and not restrictive, should not restrict the protection scope of the present application, and further includes any combination of the specific embodiments, and any equivalent transformation of the technical solution of the present application adopted by those of ordinary skilled in the art by reading the specification of the present application is covered by the claims of the present application.

Experimental methods used in the following embodiments are conventional unless otherwise specified; and materials, reagents used in the following embodiments can be obtained by commercial means unless otherwise specified.

The present application applies conventional techniques and methods used in the field of molecular biology, and those skilled in the art can adopt other conventional methods, experimental solutions and reagents of the field on the basis of technical solutions recorded in the present application, without the limitation of the specific embodiments of the present application.

### Embodiment 1 Obtaining of Soluble Tim-3 (sTim-3) Recombinant Protein and Mutant Recombinant Protein Thereof

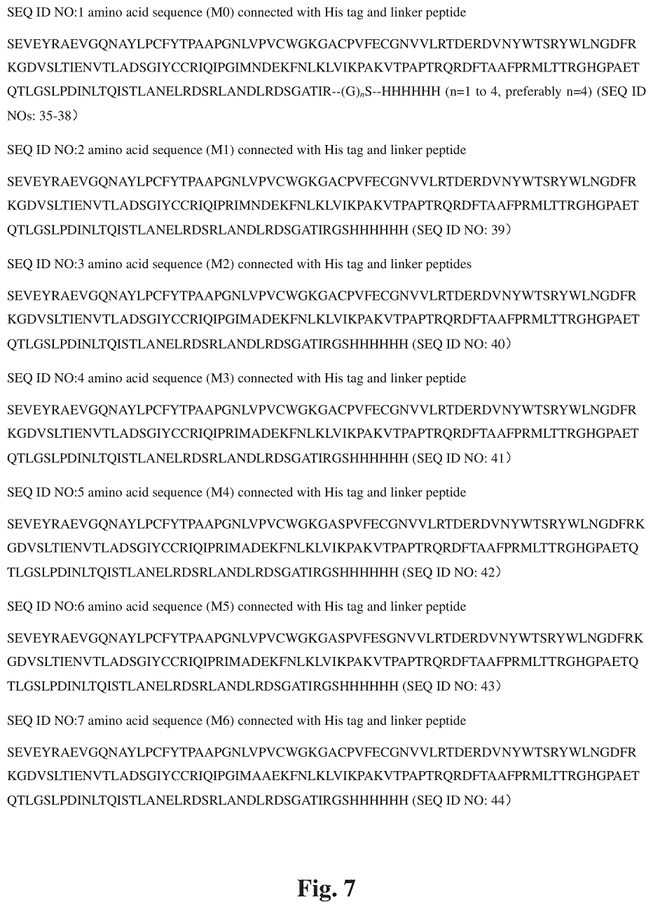

The amino acid sequence of a human Tim-3 (NP_116171.3) extracellular domain is as shown in SEQ ID NO: 1. In a human Tim-3 (NP_116171.3) extracellular domain, linker peptide (G)nS and six His fused amino acid sequence (FIG. 7), n is 1-4, preferably 4. Six amino acid sequences as shown in SEQ ID NOs: 2-7 of the mutant protein on the basis of SEQ ID NO: 1 are listed. Nucleotide sequences encoded with or without His amino acids are constructed into eukaryotic expression vectors, prokaryotic expression vectors or insect baculovirus expression vectors, and then the recombinant expression vectors are transformed into a eukaryotic, prokaryotic or insect host for expression.

In order to obtain the mutant recombinant protein with stronger biological functions, a large number of mutations of the recombinant protein are screened by a directed evolution technology, and PCR amplification is performed by using GeneMorph II random mutation PCR kit (stratagene), and target genes are constructed into the eukaryotic expression vectors, the prokaryotic expression vectors or the baculovirus expression vectors, and then the mutant recombinant expression vectors are transformed into the eukaryotic, prokaryotic or insect host for expression.

In order to obtain mutant recombinant expression plasmids with higher expression efficiency, codon optimization is performed through a host codon optimization technology. Specifically, according to the characteristics of eukaryotic, prokaryotic or insect codons of the host, the site-directed mutation is performed by recombinant PCR technology to obtain a codon-optimized target sequence, then the target sequence is digested to be cloned to the corresponding expression vectors, and then the mutant recombinant expression vectors are transformed into the eukaryotic, prokaryotic or insect host for expression.

Expression and Purification of Soluble Tim-3 and Mutant Recombinant Protein

The soluble Tim-3 recombinant protein and the mutant recombinant protein are purified through a combination of various purification methods such as affinity chromatography, ion exchange chromatography and hydrophobic chromatography. The principle of metal-chelate affinity chromatography is that some special amino acids on the surface of the protein interact with metal ions, thereby performing affinity purification on the protein, wherein a fusion tag 6×His-Tag with a combination of six His residues is relatively common (FIG. 7) and has the advantages of simple ligands, large adsorbing capacity, mild separation conditions, high universality, etc. Ion chromatography can not only carry out crude purification to effectively remove most of electrically charged host proteins and nucleic acids, but also achieve fine purification to remove trace impure proteins. Hydrophobic chromatography can not only enrich proteins but also remove most of pigment substances.

1. Constructing Primers Used by Recombinant Expression Vectors Containing Amino Acid Sequences (as shown in FIG. 7) with Six Mutant Nucleotide Sequences (SEQ ID NOs: 2-7) and Connected with His tag and Linker Peptides

The primer details are as follows:

SEQ ID NO: 2 amino acid sequence (M1) connected
with His tag and linker peptides
Primer 9181P-TIM3-F (SEQ ID NO: 11):
AGTTTAAACGGATCTCTAGCgaattcGCCGCCACCATGTTCTCCCACCTG
CC
Primer 9181P-TIM3-R (SEQ ID NO: 12):
TCGAGGTCGGGGGATCCGCGGCCGCTCAGTGATGGTGGTGGTGGTGGGAG
CCTC
Primer 9181P-L140R-F1 (SEQ ID NO: 13):
GCCAAGGTGACCCCCGCCCCCACCAGACAAAGAGATTTCACAGCCGCC
Primer 9181P-L140R-R1 (SEQ ID NO: 14):
GGCGGCTGTGAAATCTCTTTGTCTGGTGGGGGCGGGGGTCACCTTGGC
SEQ ID NO: 3 amino acid sequence (M2) connected
with His tag and linker peptides
Primer 9181P-TIM3-F (SEQ ID NO: 15):
AGTTTAAACGGATCTCTAGCgaattcGCCGCCACCATGTTCTCCCACCTG
CC
Primer 9181P-TIM3-R (SEQ ID NO: 16):
TCGAGGTCGGGGGATCCGCGGCCGCTCAGTGATGGTGGTGGTGGTGGGAG
CCTC
Primer 9181P-L140R-F1(SEQ ID NO: 17):
GCCAAGGTGACCCCCGCCCCCACCAGACAAAGAGATTTCACAGCCGCC
Primer 9181P-L140R-R1 (SEQ ID NO: 18):
GGCGGCTGTGAAATCTCTTTGTCTGGTGGGGGCGGGGGTCACCTTGGC
SEQ ID NO: 4 amino acid sequence (M3) connected
with His tag and linker peptides
Primer 9181P-TIM3-F (SEQ ID NO: 19):
AGTTTAAACGGATCTCTAGCgaattcGCCGCCACCATGTTCTCCCACCTG
CC
Primer 9181P-TIM3-R (SEQ ID NO: 20):
TCGAGGTCGGGGGATCCGCGGCCGCTCAGTGATGGTGGTGGTGGTGGGAG
CCTC
Primer 9181P-L140R-F1 (SEQ ID NO: 21):
GCCAAGGTGACCCCCGCCCCCACCAGACAAAGAGATTTCACAGCCGCC
Primer 9181P-L140R-R1 (SEQ ID NO: 22):
GGCGGCTGTGAAATCTCTTTGTCTGGTGGGGGCGGGGGTCACCTTGGC
SEQ ID NO: 5 amino acid sequence (M4) connected
with His tag and linker peptides
Primer 9181P-TIM3-F (SEQ ID NO: 23):
AGTTTAAACGGATCTCTAGCgaattcGCCGCCACCATGTTCTCCCACCTG
CC
Primer 9181P-TIM3-R (SEQ ID NO: 24):
TCGAGGTCGGGGGATCCGCGGCCGCTCAGTGATGGTGGTGGTGGTGGGAG
CCTC
Primer 9181P-L140R-F1 (SEQ ID NO: 25):
GCCAAGGTGACCCCCGCCCCCACCAGACAAAGAGATTTCACAGCCGCC
Primer 9181P-L140R-R1 (SEQ ID NO: 26):
GGCGGCTGTGAAATCTCTTTGTCTGGTGGGGGCGGGGGTCACCTTGGC
SEQ ID NO: 6 amino acid sequence (M5) connected
with His tag and linker peptides
Primer 9181P-TIM3-F (SEQ ID NO: 27):
AGTTTAAACGGATCTCTAGCgaattcGCCGCCACCATGTTCTCCCACCTG
CC
Primer 9181P-TIM3-R (SEQ ID NO: 28):
TCGAGGTCGGGGGATCCGCGGCCGCTCAGTGATGGTGGTGGTGGTGGGAG
CCTC
Primer 9181P-L140R-F1 (SEQ ID NO: 29):
GCCAAGGTGACCCCCGCCCCCACCAGACAAAGAGATTTCACAGCCGCC
Primer 9181P-L140R-R1 (SEQ ID NO: 30):
GGCGGCTGTGAAATCTCTTTGTCTGGTGGGGGCGGGGGTCACCTTGGC
SEQ ID NO: 7 amino acid sequence (M6) connected
with His tag and linker peptides
Primer 9181P-TIM3-F (SEQ ID NO: 31):
AGTTTAAACGGATCTCTAGCgaattcGCCGCCACCATGTTCTCCCACCTG
CC
Primer 9181P-TIM3-R (SEQ ID NO: 32):
TCGAGGTCGGGGGATCCGCGGCCGCTCAGTGATGGTGGTGGTGGTGGGAG
CCTC
Primer 9181P-M6-F1 (SEQ ID NO: 33):
AGGATCCAGATCCCTAGAATCATGGCCGCCGAGAAGTTTAACCTGAAGCT
GG
Primer 9181P-M6-R1 (SEQ ID NO: 34):
CCAGCTTCAGGTTAAACTTCTCGGCGGCCATGATTCTAGGGATCTGGATC
CT

2. PCR Reaction System and Procedure Used by Constructing Recombinant Expression Vectors

The 10 μl PCR reaction system is as follows:

2×Taq enzyme: 5 μl

forward primer F(10 μM): 0.4 μl

reverse primer F(10 μM): 0.4 μl

template: 1 μl

double distilled water added to: 10 μl

The PCR reaction procedure is as follows:

initial degeneration: 95° C., 5 min

degeneration: 95° C., 30 s

annealing: 55° C., 30 s

extension: 72° C., 2 min

number of cycles: 30

the last step of extension: 72° C., 5 min

storage: keep at 16° C.

3. Construction of Recombinant Expression Vectors, Transient Transfection, Expression and Purification

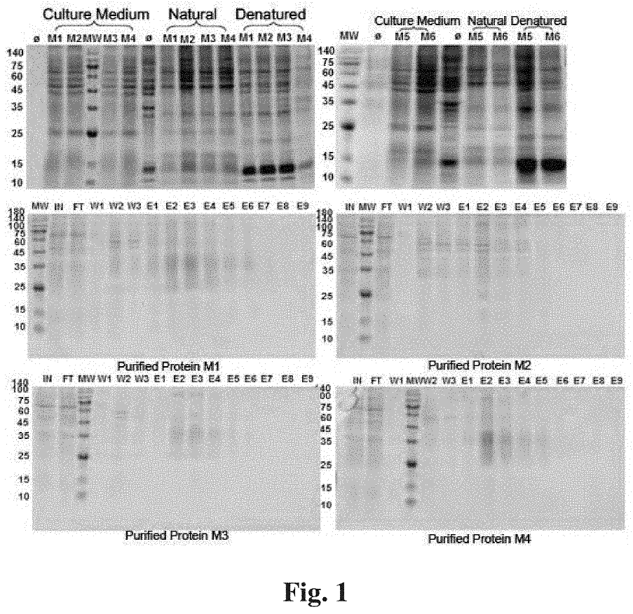

pATX2 expression vectors were constructed, transiently transfected into 80 ml of HEK293 cells, and on day 6 after transfection, 1.5 ml of cell culture media and cells were collected. The expressed target recombinant proteins were purified, and the culture medium, natural proteins, denatured proteins and purified proteins were identified by SDS-PAGE and WB. The results are shown in FIG. 1. The concentrations of the purified recombinant proteins M1, M2, M3, M4, M5 and M6 were 0.1 mg/ml, 0.05 mg/ml, 0.05 mg/ml, 0.1 mg/ml, 0.22 mg/ml, 0.11 mg/ml and 0.17 mg/ml, respectively. The purified recombinant proteins were freeze-dried and preserved.

### Embodiment 2 Soluble Tim-3 Inhibits Activation of Monocytes to Release Inflammatory Factors

1. Dose-Dependent Effect of Soluble Tim-3 on the Ability of TNF-α Cytokine Secretion of the Monocytes

The primary monocytes were separated from peripheral blood of healthy people by magnetic beads. When the cells were stimulated by 1 μg/ml LPS, the primary monocytes were treated with soluble Tim-3 at a concentration of 10, 20, 40 and 80 ng/ml at the same time, and 1 μg/ml LPS stimulation was performed for 24 h; or the monocytes were stimulated by 1 μg/ml LPS for 30 min firstly, and then the primary monocytes were intervened by soluble Tim-3 at the concentration of 10, 20, 40 and 80 ng/ml, and 1 μg/ml LPS stimulation was performed for 24 h in total. The cell supernatant was collected, and the level of TNF-α in the cell supernatant was detected by ELISA, in which the instrument used was American Bio-Rad iMark absorbance microplate reader, the reagent used was TNF-α ELISA kit, and the specific steps were performed according to the specification.

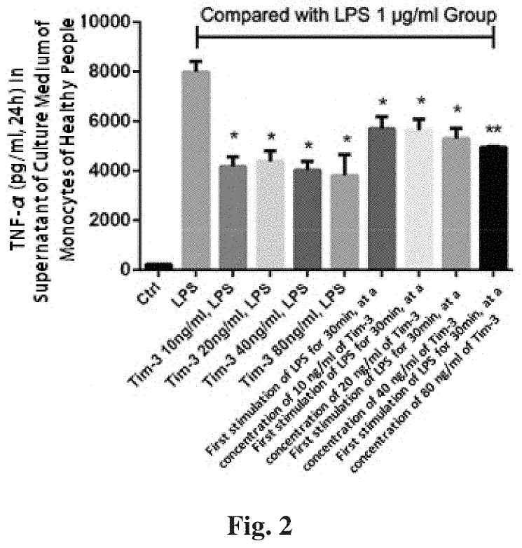

As shown in FIG. 2, compared with LPS group, the soluble Tim-3 significantly inhibits the ability of the secretion of cytokine TNF-α of LPS-stimulated monocytes, with significant differences and a dose-dependent effect. Besides, the simultaneous intervention by soluble Tim-3 and LPS has a better inhibitory effect of the cytokine TNF-α secretion than stimulation by LPS for 30 minutes firstly, and the highest dose of 80 ng/ml soluble Tim-3 has the best TNF-α inhibitory effect.

2. Effect of Soluble Tim-3 on the Ability of HMGB1 Secretion of the Monocytes

The primary monocytes were separated from peripheral blood of healthy people by magnetic beads. When the cells were stimulated by 1 μg/ml LPS, the primary monocytes were treated with soluble Tim-3 at a concentration of 20 ng/ml at the same time, and 1 μg/ml LPS stimulation was performed for 24 h. The cell supernatant was collected, and the level of HMGB1 in the cell supernatant was detected by ELISA, in which the instrument used was American Bio-Rad iMark absorbance microplate reader, the reagent used was HMGB1 ELISA kit, and the specific steps were performed according to the specification.

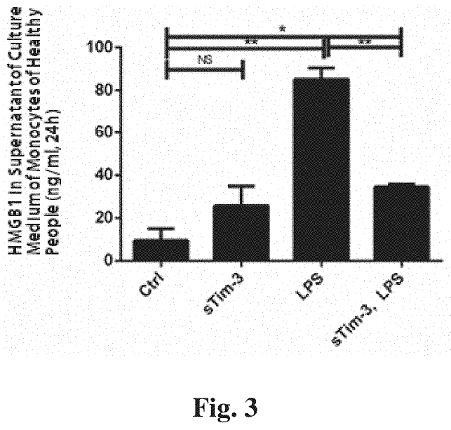

As shown in FIG. 3, compared with LPS group, soluble Tim-3 significantly inhibited the ability of the secretion of the inflammatory mediator HMGB1 of LPS-stimulated monocytes, with significant differences.

### Embodiment 3 Effect of Soluble Tim-3 on Liver Damage of Liver Failure

1. Expression Levels of Soluble Tim-3 and Membraned Tim-3 of Monocytes in Patients with Acute on Chronic Liver Failure

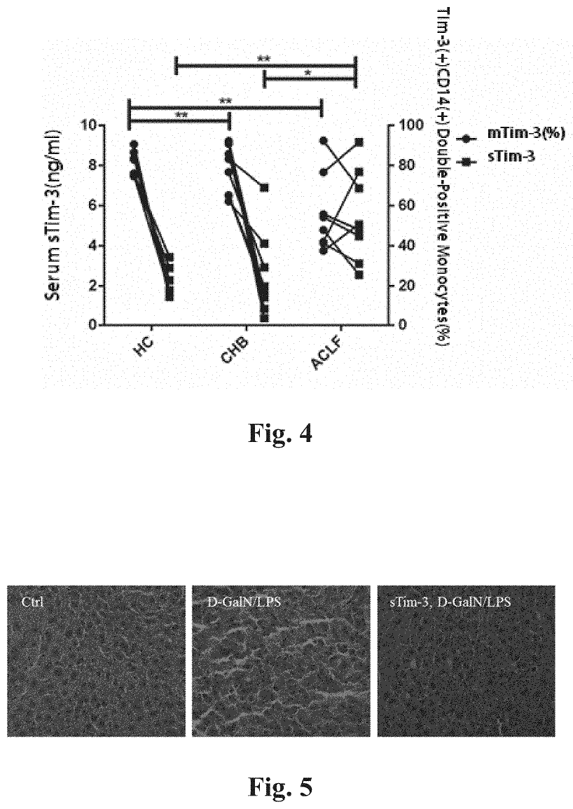

Peripheral blood of the patients with acute on chronic liver failure (ACLF, n=8), patients with chronic hepatitis B (CHB, n=8) and health controls (HC, n=8) were collected. As for peripheral blood samples, an anti-human CD14-APC flow antibody and an anti-human Tim-3-PE flow antibody were evenly mixed with the 100 μl of blood samples, and the mixture was incubated at room temperature in the dark for 15 min. Then 1 ml of red blood cell lysis buffer was added and evenly mixed, and the obtained mixture was incubated at room temperature for 10 min After the incubation, the mixture was centrifuged, washed with washing liquid, and resuspended, and then prepared for testing on the machine. The flow analysis software was started, 100,000 cells were collected, and the positive percentage of Tim-3 of CD14 positive monocytes was analyzed. The results in FIG. 4 show that the plasma soluble Tim-3 levels of the patients with chronic acute liver failure (ACLF, n=8), the patients with chronic hepatitis B (CHB, n=8) and healthy controls (HC, n=8) were increased significantly along with disease progression, while the membraned Tim-3 (mTim-3) levels of the monocytes were decreased significantly along with disease progression.

2. Effect of Soluble Tim-3 on Liver Damage in Mice with Acute Liver Failure

In the model group, the mice model with acute liver failure was induced by the combination of D-galactosamine (D-GalN) with endotoxin (LPS). The soluble Tim-3 treatment group was injected with soluble Tim-3 through tail veins 30 min after the acute hepatic failure model was established, while the control group was injected with normal saline through tail veins. The C57BL/6 mice used are: male, 6-8 weeks of age, and each of 18-20 g. Liver histology findings obtained through HE-stained detection show that sTim-3 significantly improved liver cell necrosis and had a protective effect on the model (FIG. 5).

### Embodiment 4 Inhibitory Effect of Mutant and Non-mutant sTim-3 on TNF-α Secretion from Monocytes

Protein sequences of mutant sTim-3 of the embodiment were as shown in SEQ ID NOs: 2-7 (amino acid sequences connected with His tag and linker peptides, M1, M2, M3, M4, M5 and M6). Both M0 and sTim-3 were non-mutant sTim-3 protein sequences, wherein M0 was recombinantly expressed in combination with mutant sTim-3 which is a commercially available recombinant protein.

The primary monocytes were separated from peripheral blood of healthy people by Magnetic beads. When cells were stimulated by 1 μg/ml LPS, the primary monocytes were treated with soluble Tim-3 at a concentration of 20 ng/ml 30 min in advance and then 1 μg/ml LPS stimulation was performed for 24 h. The cell supernatant was collected, and the level of TNF-α was detected by ELISA, in which the instrument used was American Bio-Rad iMark absorbance microplate reader, the reagent used was TNF-α ELISA kit, and the specific steps were performed according to the specification.

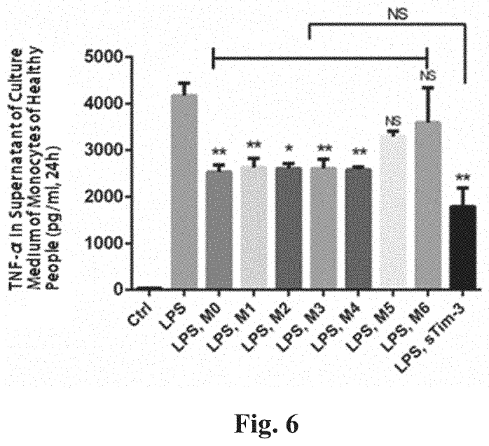

As shown in FIG. 6, compared with LPS group, mutant sTim-3 proteins (M1, M2, M3 and M4), M0 and sTim-3 all significantly inhibited the ability of the secretion of cytokine TNF-α of LPS-stimulated monocytes, with significant differences, while neither M5 nor M6 significantly inhibits the ability of the secretion of cytokine TNF-α of LPS-stimulated monocytes, with significant differences. Compared with commercial sTim-3, the recombinant proteins (M0, M1, M2, M3, M4, M5 and M6) show no significant difference in ability of inhibiting the secretion of TNF-α.

What is claimed is:

1. Use of Soluble Tim-3 recombinant protein in preparing a drug with the function of regulating monocytes or a drug with the function of enhancing tumor immune response, wherein the amino acid sequence of the soluble Tim-3 recombinant protein is as shown in SEQ ID NO: 1.

2. Use according to claim 1, wherein the drug with the function of regulating monocytes can be used for inhibiting overactivity of the monocytes in patients with inflammation, and the drug with the function of enhancing tumor immune response can be used for enhancing immune response in cancer patients to avoid host's immune escape.

3. A purification method of soluble Tim-3 recombinant protein, comprising the following steps: performing fluid exchanging on cell culture solutions of eukaryotic CHO cells, prokaryotic E. coli and insect baculovirus expression system containing recombinant human soluble Tim-3 through microfiltration clarification and ultrafiltration concentration, and then passing through cation and anion chromatographic column, molecular sieve chromatographic column, hydrophobic chromatographic column and/or affinity chromatographic column to obtain the soluble Tim-3 recombinant protein with enough purity, wherein the amino acid sequence of the soluble Tim-3 recombinant protein is as shown in SEQ ID NO: 1.

4. A soluble Tim-3 recombinant mutant protein, wherein the mutant protein is a soluble Tim-3 recombinant mutant protein obtained in the mode that: on the basis of a gene sequence corresponding to human Tim-3 (NP_116171.3) extracellular domain amino acid (SEQ ID NO: 1), mutant genes are obtained by screening through a directed evolutionary technology, and then expressed, wherein the mutant sites of the mutant genes do not involve conserved sequences among species, but occur in non-conservative sequence positions.

5. A soluble Tim-3 recombinant mutant protein, wherein the amino acid sequence of the soluble Tim-3 recombinant mutant protein is one of the sequences as shown in SEQ ID NOs: 2-7.

6. The soluble Tim-3 recombinant mutant protein according to claim 4, wherein the soluble Tim-3 recombinant mutant protein further comprises a His tag sequence preceded by a linker peptide.

7. The soluble Tim-3 recombinant mutant protein according to claim 6, wherein the His tag sequence is HHHHHH, and the linker peptide is (G)nS, n=1-4.

8. A preparation method of the soluble Tim-3 recombinant mutant protein according to claim 4, wherein on the basis of the gene sequence corresponding to the human Tim-3 (NP_116171.3) extracellular domain amino acid (SEQ ID NO: 1), mutant genes are obtained by screening through a directed evolutionary technology, and then amino acids are expressed into the soluble Tim-3 recombinant mutant protein.

9. Use of the soluble Tim-3 mutant protein according to claim 4 in preparing a drug with the function of regulating monocytes or a drug with the function of enhancing tumor immune response.

10. The use according to claim 9, wherein the drug with the function of regulating monocytes can be used for inhibiting overactivity of the monocytes in patients with inflammation, and the drug with the function of enhancing tumor immune response can be used for enhancing immune response in cancer patients to avoid host's immune escape.

11. The soluble Tim-3 recombinant mutant protein according to claim 5, wherein the soluble Tim-3 recombinant mutant protein further comprises a His tag sequence preceded by a linker peptide.

12. The soluble Tim-3 recombinant mutant protein according to claim 11, wherein the His tag sequence is HHHHHH, and the linker peptide is (G)nS, n=1-4.

13. A preparation method of the soluble Tim-3 recombinant mutant protein according to claim 5, wherein on the basis of the gene sequence corresponding to the human Tim-3 (NP_116171.3) extracellular domain amino acid (SEQ ID NO: 1), mutant genes are obtained by screening through a directed evolutionary technology, and then amino acids are expressed into the soluble Tim-3 recombinant mutant protein.

14. Use of the soluble Tim-3 mutant protein according to claim 5 in preparing a drug with the function of regulating monocytes or a drug with the function of enhancing tumor immune response.

15. The use according to claim 14, wherein the drug with the function of regulating monocytes can be used for inhibiting overactivity of the monocytes in patients with inflammation, and the drug with the function of enhancing tumor immune response can be used for enhancing immune response in cancer patients to avoid host's immune escape.

16. A preparation method of the soluble Tim-3 recombinant mutant protein according to claim 5, wherein on the basis of the gene sequence corresponding to the human Tim-3 (NP_116171.3) extracellular domain amino acid (SEQ ID NO: 1), mutant genes are obtained by screening through a directed evolutionary technology, and then amino acids are expressed into the soluble Tim-3 recombinant mutant protein.

17. A preparation method of the soluble Tim-3 recombinant mutant protein according to claim 6, wherein on the basis of the gene sequence corresponding to the human Tim-3 (NP_116171.3) extracellular domain amino acid (SEQ ID NO: 1), mutant genes are obtained by screening through a directed evolutionary technology, and then amino acids are expressed into the soluble Tim-3 recombinant mutant protein.

18. A preparation method of the soluble Tim-3 recombinant mutant protein according to claim 7, wherein on the basis of the gene sequence corresponding to the human Tim-3 (NP_116171.3) extracellular domain amino acid (SEQ ID NO: 1), mutant genes are obtained by screening through a directed evolutionary technology, and then amino acids are expressed into the soluble Tim-3 recombinant mutant protein.

19. A preparation method of the soluble Tim-3 recombinant mutant protein according to claim 11, wherein on the basis of the gene sequence corresponding to the human Tim-3 (NP_116171.3) extracellular domain amino acid (SEQ ID NO: 1), mutant genes are obtained by screening through a directed evolutionary technology, and then amino acids are expressed into the soluble Tim-3 recombinant mutant protein.

20. A preparation method of the soluble Tim-3 recombinant mutant protein according to claim 12, wherein on the basis of the gene sequence corresponding to the human Tim-3 (NP_116171.3) extracellular domain amino acid (SEQ ID NO: 1), mutant genes are obtained by screening through a directed evolutionary technology, and then amino acids are expressed into the soluble Tim-3 recombinant mutant protein.


SEQUENCE LISTING (44 sequences)

SEQ ID NO: 1 - protein, 179 aa, Artificial Sequence
  soluble Tim-3 recombinant protein
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIR

SEQ ID NO: 2 - protein, 179 aa, Artificial Sequence
  soluble Tim-3 recombinant mutant protein
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPRIMNDEKFNLKLVIKPAKVTPAPTRQRDFTAAFPRMLTTRGHGPAETQTLGSLPDINLTQISTLANELRDSRLANDLRDSGATIR

SEQ ID NO: 3 - protein, 179 aa, Artificial Sequence
  soluble Tim-3 recombinant mutant protein
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMADEKFNLKLVIKPAKVTPAPTRQRDFTAAFPRMLTTRGHGPAETQTLGSLPDINLTQISTLANELRDSRLANDLRDSGATIR

SEQ ID NO: 4 - protein, 179 aa, Artificial Sequence
  soluble Tim-3 recombinant mutant protein
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPRIMADEKFNLKLVIKPAKVTPAPTRQRDFTAAFPRMLTTRGHGPAETQTLGSLPDINLTQISTLANELRDSRLANDLRDSGATIR

SEQ ID NO: 5 - protein, 179 aa, Artificial Sequence
  soluble Tim-3 recombinant mutant protein
SEVEYRAEVGQNAYLPCFYTPAAPGNLVPVCWGKGASPVFECGNVVLRTDERDVNYWTSRYWLNGDFRKGDVSLTIENVTLADSGIYCCRIQIPRIMADEKFNLKLVIKPAKVTPAPTRQRDFTAAFPRMLTTRGHGPAETQTLGSLPDINLTQISTLANELRDSRLANDLRDSGATIR

SEQ ID NO: 6 - protein, 179 aa, Artificial Sequence
  soluble Tim-3 recombinant mutant protein
SEVEYRAEVGQNAYLPCFYTPAAPGNLVPVCWGKGASPVFESGNVVLRTDERDVNYWTSRYWLNGDFRKGDVSLTIENVTLADSGIYCCRIQIPRIMADEKFNLKLVIKPAKVTPAPTRQRDFTAAFPRMLTTRGHGPAETQTLGSLPDINLTQISTLANELRDSRLANDLRDSGATIR

SEQ ID NO: 7 - protein, 179 aa, Artificial Sequence
  soluble Tim-3 recombinant mutant protein
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMAAEKFNLKLVIKPAKVTPAPTRQRDFTAAFPRMLTTRGHGPAETQTLGSLPDINLTQISTLANELRDSRLANDLRDSGATIR

SEQ ID NO: 8 - protein, 6 aa, Artificial Sequence
  his tag sequence
HHHHHH

SEQ ID NO: 9 - protein, 4 aa, Artificial Sequence
  linker peptide
GGGS

SEQ ID NO: 10 - protein, 5 aa, Artificial Sequence
  linker peptides
GGGGH

SEQ ID NO: 11 - DNA, 52 bp, Artificial Sequence
  Primer 9181P-TIM3-F
agtttaaacggatctctagcgaattcgccgccaccatgttctcccacctgcc

SEQ ID NO: 12 - DNA, 54 bp, Artificial Sequence
  Primer 9181P-TIM3-R
tcgaggtcgggggatccgcggccgctcagtgatggtggtggtggtgggagcctc

SEQ ID NO: 13 - DNA, 48 bp, Artificial Sequence
  Primer 9181P-L140R-F1
gccaaggtgacccccgcccccaccagacaaagagatttcacagccgcc

SEQ ID NO: 14 - DNA, 48 bp, Artificial Sequence
  Primer 9181P-L140R-R1
ggcggctgtgaaatctctttgtctggtgggggcgggggtcaccttggc

SEQ ID NO: 15 - DNA, 52 bp, Artificial Sequence; identical to SEQ ID NO: 11
  Primer 9181P-TIM3-F

SEQ ID NO: 16 - DNA, 54 bp, Artificial Sequence; identical to SEQ ID NO: 12
  Primer 9181P-TIM3-R

SEQ ID NO: 17 - DNA, 48 bp, Artificial Sequence; identical to SEQ ID NO: 13
  Primer 9181P-L140R-F1

SEQ ID NO: 18 - DNA, 48 bp, Artificial Sequence; identical to SEQ ID NO: 14
  Primer 9181P-L140R-R1

SEQ ID NO: 19 - DNA, 52 bp, Artificial Sequence; identical to SEQ ID NO: 11
  Primer 9181P-TIM3-F

SEQ ID NO: 20 - DNA, 54 bp, Artificial Sequence; identical to SEQ ID NO: 12
  Primer 9181P-TIM3-R

SEQ ID NO: 21 - DNA, 48 bp, Artificial Sequence; identical to SEQ ID NO: 13
  Primer 9181P-L140R-F1

SEQ ID NO: 22 - DNA, 48 bp, Artificial Sequence; identical to SEQ ID NO: 14
  Primer 9181P-L140R-R1

SEQ ID NO: 23 - DNA, 52 bp, Artificial Sequence; identical to SEQ ID NO: 11
  Primer 9181P-TIM3-F

SEQ ID NO: 24 - DNA, 54 bp, Artificial Sequence; identical to SEQ ID NO: 12
  Primer 9181P-TIM3-R

SEQ ID NO: 25 - DNA, 48 bp, Artificial Sequence; identical to SEQ ID NO: 13
  Primer 9181P-L140R-F1

SEQ ID NO: 26 - DNA, 48 bp, Artificial Sequence; identical to SEQ ID NO: 14
  Primer 9181P-L140R-R1

SEQ ID NO: 27 - DNA, 52 bp, Artificial Sequence; identical to SEQ ID NO: 11
  Primer 9181P-TIM3-F

SEQ ID NO: 28 - DNA, 54 bp, Artificial Sequence; identical to SEQ ID NO: 12
  Primer 9181P-TIM3-R

SEQ ID NO: 29 - DNA, 48 bp, Artificial Sequence; identical to SEQ ID NO: 13
  Primer 9181P-L140R-F1

SEQ ID NO: 30 - DNA, 48 bp, Artificial Sequence; identical to SEQ ID NO: 14
  Primer 9181P-L140R-R1

SEQ ID NO: 31 - DNA, 52 bp, Artificial Sequence; identical to SEQ ID NO: 11
  Primer 9181P-TIM3-F

SEQ ID NO: 32 - DNA, 54 bp, Artificial Sequence; identical to SEQ ID NO: 12
  Primer 9181P-TIM3-R

SEQ ID NO: 33 - DNA, 52 bp, Artificial Sequence
  Primer 9181P-M6-F1
aggatccagatccctagaatcatggccgccgagaagtttaacctgaagctgg

SEQ ID NO: 34 - DNA, 52 bp, Artificial Sequence
  Primer 9181P-M6-R1
ccagcttcaggttaaacttctcggcggccatgattctagggatctggatcct

SEQ ID NO: 35 - protein, 187 aa, Artificial Sequence
  amino acid sequence (M0) connected with His tag and linker peptide
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRGSHHHHHH

SEQ ID NO: 36 - protein, 188 aa, Artificial Sequence
  amino acid sequence (M0) connected with His tag and linker peptide
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRGGSHHHHHH

SEQ ID NO: 37 - protein, 189 aa, Artificial Sequence
  amino acid sequence (M0) connected with His tag and linker peptide
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRGGGSHHHHHH

SEQ ID NO: 38 - protein, 190 aa, Artificial Sequence
  amino acid sequence (M0) connected with His tag and linker peptide
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRGGGGSHHHHHH

SEQ ID NO: 39 - protein, 187 aa, Artificial Sequence
  amino acid sequence (M1) connected with His tag and linker peptide
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPRIMNDEKFNLKLVIKPAKVTPAPTRQRDFTAAFPRMLTTRGHGPAETQTLGSLPDINLTQISTLANELRDSRLANDLRDSGATIRGSHHHHHH

SEQ ID NO: 40 - protein, 187 aa, Artificial Sequence
  amino acid sequence (M2) connected with His tag and linker peptides
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMADEKFNLKLVIKPAKVTPAPTRQRDFTAAFPRMLTTRGHGPAETQTLGSLPDINLTQISTLANELRDSRLANDLRDSGATIRGSHHHHHH

SEQ ID NO: 41 - protein, 187 aa, Artificial Sequence
  amino acid sequence (M3) connected with His tag and linker peptide
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPRIMADEKFNLKLVIKPAKVTPAPTRQRDFTAAFPRMLTTRGHGPAETQTLGSLPDINLTQISTLANELRDSRLANDLRDSGATIRGSHHHHHH

SEQ ID NO: 42 - protein, 187 aa, Artificial Sequence
  amino acid sequence (M4) connected with His tag and linker peptide
SEVEYRAEVGQNAYLPCFYTPAAPGNLVPVCWGKGASPVFECGNVVLRTDERDVNYWTSRYWLNGDFRKGDVSLTIENVTLADSGIYCCRIQIPRIMADEKFNLKLVIKPAKVTPAPTRQRDFTAAFPRMLTTRGHGPAETQTLGSLPDINLTQISTLANELRDSRLANDLRDSGATIRGSHHHHHH

SEQ ID NO: 43 - protein, 187 aa, Artificial Sequence
  amino acid sequence (M5) connected with His tag and linker peptide
SEVEYRAEVGQNAYLPCFYTPAAPGNLVPVCWGKGASPVFESGNVVLRTDERDVNYWTSRYWLNGDFRKGDVSLTIENVTLADSGIYCCRIQIPRIMADEKFNLKLVIKPAKVTPAPTRQRDFTAAFPRMLTTRGHGPAETQTLGSLPDINLTQISTLANELRDSRLANDLRDSGATIRGSHHHHHH

SEQ ID NO: 44 - protein, 187 aa, Artificial Sequence
  amino acid sequence (M6) connected with His tag and linker peptide
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMAAEKFNLKLVIKPAKVTPAPTRQRDFTAAFPRMLTTRGHGPAETQTLGSLPDINLTQISTLANELRDSRLANDLRDSGATIRGSHHHHHH